(19) United States
(12) Patent Application Publication
(10) Pub. No.: US 2022/0211066 A1
(43) Pub. Date: Jul. 7, 2022

(54) A FAT COMPOSITION ENRICHED WITH MEDIUM-AND LONG-CHAIN TRIACYLGLYCEROL AND ITS PREPARATION PROCESS

(71) Applicant: JIANGNAN UNIVERSITY, Wuxi, Jiangsu (CN)

(72) Inventors: Xingguo WANG, Wuxi, Jiangsu (CN); WEI WEI, Wuxi, Jiangsu (CN); Qingzhe JIN, Wuxi, Jiangsu (CN); Tinglan YUAN, Wuxi, Jiangsu (CN)

(21) Appl. No.: 17/603,776

(22) PCT Filed: May 20, 2020
(86) PCT No.: PCT/CN2020/091251

§ 371 Date: Oct. 14, 2021

(30) Foreign Application Priority Data
Apr. 18, 2019 (CN) 201910311784.2

(51) Int. Cl. A23D 9/02; A23L 33/115

(52) U.S. Cl. CPC A23D 9/02; A23L 33/115

(57) ABSTRACT

The invention provides a fat composition enriched with medium-and long-chain triacylglycerol and its preparation process. As for fatty acid composition, the content of medium-chain fatty acids and long-chain fatty acids reach above 99%, the content of short-chain fatty acids is less than 1%, and the mass ratio of long-chain fatty acids to medium-chain fatty acids is from 1.2:1 to 3:1. As for triacylglycerol composition, the triacylglycerol containing one medium-chain fatty acid and two long-chain fatty acids makes up 50-90% of the total triacylglycerol; the triacylglycerol containing two medium-chain fatty acids and one long-chain fatty acid makes up 3-35% of the total triacylglycerol; the triacylglycerol containing three medium-chain fatty acids makes up 0-10% of the total triacylglycerol. The fat composition has better oxidation stability and fat digestion and absorption rate, meeting the needs of particular groups of infants and young children.

### FIELD OF THE INVENTION

The present invention relates to a fat composition enriched with medium-and long-chain triacylglycerol, belonging to the technical field of fat.

### BACKGROUND OF THE INVENTION

Long-chain triacylglycerols (LCT) are usually rich in the vast majority of edible oils and fats, providing essential fatty acids for the body's normal development. However, after LCT was hydrolyzed, novel triacylglycerols are resynthesized in the small intestinal villus cells. The resynthesized triacylglycerols are combined with soluble proteins and then enter the blood system through the lymphatic system in the form of chylomicron through the thoracic duct. Finally, they are transported to the liver, where the long-chain fatty acids can enter mitochondria for oxidative metabolism relying on the carnitine carrier. The relatively slow rate of hydrolysis and clearance in serum has significant effects on the metabolic health of people with liver dysfunction, abnormal metabolism and infants.

The relative molecular weight of medium-chain triacylglycerol (MCT) is lower than that of LCT, and its water solubility is much higher than that of LCT, so the hydrolysis of MCT is faster and more complete than that of LCT. MCT passes through the small intestinal villus cells quickly, without forming chylomicron, and directly enters the portal vein for transport through the circulatory system to the liver, where it also directly enters the mitochondria for oxidative metabolism without relying on carnitine carriers. Its digestion and absorption rate are four times that of LCT, metabolic rate was 10 times that of LCT, but it does not contain essential fatty acids for humans. Moreover, it is easy to cross the blood-brain barrier. The caprylic acid has central nervous system toxicity, which would cause intoxication after excessive intake of a large number of ketones, so the MCT should not be consumed for a long time from the point of view of nutrition. The simple physical mixing of MCT and LCT can contain both MCT and LCT, but it still cannot overcome its own metabolic shortcomings. Compared with physical-mixed MCT/LCT, medium-and long-chain triacylglycerol (MLCT) had better oxidation stability, faster hydrolysis release rate, oxidation rate, and plasma clearance rate. It has the advantages of reducing plasma triacylglycerol and cholesterol content, weakening catabolism of protein, promoting positive nitrogen balance, improving the bioavailability of nutrients, inhibiting fat accumulation in the body, improving immune function without affecting the function of the reticuloendothelial system.

Existing products of MLCT structural lipids are mainly used in parenteral fat emulsion injection, which are suitable to supply appropriate calories and essential fatty acids for patients requiring high calories (such as tumours and other malignant diseases), with kidney damage or protein restriction, and who cannot absorb nutrients through gastrointestinal tract for some reason. But the products are not edible, and their composition does not satisfy the nutrition required for infant growth and is not suitable for infant feeding.

The patent (CN 145425 A) published a kind of fat composition containing medium-chain fatty acids for cooking, in which the mass ratio of triacylglycerols containing two medium-chain fatty acids in all triacylglycerols is 1-20%; in caprylic acid and capric acid were preferred for medium-chain fatty acids, and fatty acids with 14-22 carbon atoms were preferred for long-chain fatty acids. The patent is a composition invented for cooking, the components of which are not conducive to infant digestion, absorption, and metabolism, and its products are not suitable for infant food.

The patent (CN 103891920 A) published a kind of lipid composition containing MLCT, in which the mass ratio of triacylglycerols containing one medium-chain fatty acid is 1-90%, the mass ratio of triacylglycerols containing two medium-chain fatty acids is lower than 1%. Fatty acids composition and distribution and triacylglycerol structure are not conducive to the digestion and absorption of fatty acids for particular groups of infants and young children. It cannot meet their needs of growth and development and is not suitable for infant consumption.

Fat is the primary energy source for infant growth (about 50%), and its fatty acid composition and triacylglycerol structure plays a crucial role in infant growth and development. However, infants are at a unique growth stage with an immature digestion and absorption system, which requires greater efficiency fat. Therefore, it is necessary to study a kind of MLCT whose composition and structure are conducive to infants' digestion and absorption and meet the needs of infants and other special groups.

### SUMMARY OF THE INVENTION

The object of the present invention is to provide a fat composition enriched with MLCT, which has better oxidation stability and fat digestion and absorption rate that meets the needs of particular groups of infants and young children.

Another purpose of the invention is to provide a preparation process of the fat composition, which is clever in conception, simple in operation, low in cost, and suitable for industrial production.

The fat composition contains long-chain fatty acids and medium-chain fatty acids on the same glycerol molecular.

The invention provides a fat composition containing MLCT. In terms of fatty acid composition, the content of medium-chain fatty acids and long-chain fatty acids reaches above 99%, the content of short-chain fatty acids is less than 1%, and the mass ratio of long-chain fatty acids to medium-chain fatty acids is from 1.2:1 to 3:1. Moreover, there are not only medium-chain fatty acids but also long-chain fatty acids at the sn-2 position, where the content of saturated fatty acids is more than 60%.

In terms of triacylglycerol composition, the triacylglycerol containing one medium-chain fatty acid and two long-chain fatty acids makes up 50-90% of the total triacylglycerol, the triacylglycerol containing two medium-chain fatty acids and one long-chain fatty acid makes up 3-35% of the total triacylglycerol, the triacylglycerol containing three medium-chain fatty acids makes up 0-10% of the total triacylglycerol.

Furthermore, the short-chain fatty acids include at most 6 carbon atoms; the medium-chain fatty acid include 6-12 carbon atoms, preferring 10-12 carbon atoms; the long-chain fatty acids include 14-24 carbon atoms, preferring 16-18 carbon atoms.

Furthermore, the medium-chain fatty acids include lauric acid (La).

Furthermore, the long-chain fatty acids include palmitic acid (P), oleic acid (o), linoleic acid (L).

Furthermore, the triacylglycerols include LaPO (12:0-16:0-18:1), LaOO (12:0-18:1-18:1), LaOL (12:0-18:1-18:2), LaLL (12:0-18:2-18:2), and LaPL (12:0-16:0-18:2).

Furthermore, LaPO makes up 10-60% of the fat composition; LaOO makes up 5-40% of the fat composition; LaOL makes up 5-30% of the fat composition; LaLL makes up 0-10% of the fat composition; LaPL makes up 0-10% of the fat composition.

Preferably, LaPO makes up 10-45% of the fat composition; LaOO makes up 5-25% of the fat composition; LaOL makes up 5-20% of the fat composition; LaLL makes up 0-5% of the fat composition; LaPL makes up 0-5% of the fat composition.

The medium-chain fatty acids and long-chain fatty acids are derived from one or more fats of vegetable oil or animal oil or its extracts or milk fat. Preferably, the sources of short-chain fatty acids and medium-chain fatty acids are milk fats, or plant oils such as palm kernel oil, coconut oil; the sources of long-chain fatty acids are palm oil, rapeseed oil, high oleic acid sunflower oil, rice bran oil, algal oil, lard, sheep oil, fish oil, and other animal and vegetable oils, microbial oils or milk fats or the modified fat obtained from these oils.

The fat composition can be used as additives in food, preferably as an additive in food for infants and children.

Furthermore, the fat composition can be added with a 15-50% concentration to infant formula, follow-on formula, or infant food.

The invention also includes a process of preparing the fat composition. The process includes mixing the medium-chain triacylglycerol with the long-chain fatty acids as the ratio of 1:2-1:5 (mol/mol), or mixing long-chain triacylglycerol with the medium-chain fatty acids as the ratio of 1:0.5-1:2 (mol/mol), adding lipase with a mass ratio of 4-14% to substrate at 40-65° C., and reacting for 0.5-10 h under solvent-free conditions.

Alternatively, the process includes mixing the medium-chain triacylglycerol with the long-chain triacylglycerol as the mass ratio of 1:0.5-1:3, adding lipase with a mass ratio of 4-14% to substrate at 40-65° C., and reacting for 0.5-12 h under solvent-free conditions.

Preferably, the medium-chain triacylglycerols include one or more kinds of fats such as coconut oil, coconut oil extract, palm kernel oil, palm kernel oil extract, laurin, tricaprylin or tricaprin.

Preferably, the medium-chain triacylglycerols include the glycerides containing o caprylic acid, or capric acid or lauric acid.

Preferably, the medium-chain fatty acids include one or more fatty acids such as caprylic acid, capric acid, lauric acid.

Preferably, the reaction was carried out at 200-500 r/min.

Preferably, the long-chain fatty acids include one or more fatty acids such as myristic acid, palmitic acid, oleic acid, linoleic acid, stearic acid, linolenic acid, arachidonic acid, eicosapentaenoic acid, docosapentaenoic acid, docosahexaenoic acid.

Preferably, the long-chain triacylglycerols include fish oil, lard oil, or extracts.

Preferably, the lipases include one or more of immobilized enzyme NS 40086, Lipozyme TL IM, Lipozyme RM IM, Lipozyme 435, Novozym 435, Lipase PS, Lipase AK, Lipase AH, and Lipase AYS.

The invention has the following advantages: compared with the existing medium and long chain triacylglycerol fat composition, the fat composition enriched with medium and long chain triacylglycerol of this invention has better digestion and absorption rate, better oxidation stability, more qualitied fatty acids and triacylglycerol structure, which is more conductive to the fat digestion, absorption and metabolism in infants, more suitable for infants consumption and can better meet the requirements of infants for rapid growth and development.

The invention also provides a process of preparing the fat composition enriched with medium and long chain triacylglycerol. On one hand, the fatty acid composition and triacylglycerol structure of the products are more conducive to the digestion, absorption and metabolism of fat in infants. On the other hand, in this method, the selection of raw materials correctly and use of them reasonably made the preparation method efficient, high utilization ratio of raw material, and less impurities in the product, in which the triglyceride content is more than 90%, and the medium-long chain triglyceride content is more than 80%. At the same time, the reaction proceeds at room temperature and under solvent-free system, the green preparation method has qualitied products, simple operation, and is suitable for industrial production.

### DESCRIPTION OF THE DRAWINGS

In order to illustrate of this invention, the preparation of the appended drawings used in the below embodiments is described. Obviously, those skilled in the art will appreciate that other drawings may be obtained according to these without creative labour.

FIG. 1 shows the change curves of digestibility of embodiments of the present invention (1, 2, and 3) and reference (4) as well as human milk in the in vitro simulated gastric digestion (A) and intestinal digestion (B) systems of infants.

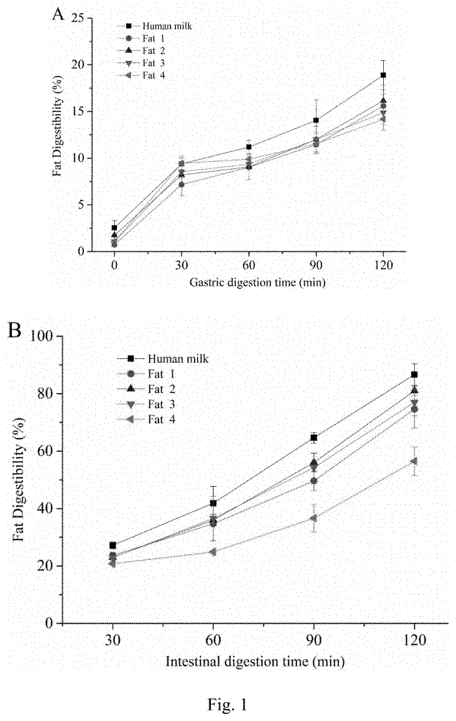

### DESCRIPTION OF THE PREFERRED EMBODIMENTS

The following examples serve to illustrate the application of this invention in combination with the appended drawings of the specification. It is obvious that the preferred embodiments of this invention are illustrated but should not be limited by the following examples. Those skilled in this art will also appreciate that all other examples obtained based on this invention without creative work shall fall within the scope of protection of this invention.

Stated in more detailed process terms, some of the more preferred embodiments of the invention may be illustrated but should not be limited by the following additional exemplary versions of the herein disclosed processes. The process of this invention can also be implemented in other ways different from those described herein. Those skilled in this art will also make similar promotions without violating the connotation of the present invention.

“An embodiment” or “an example” herein means that a particular feature, structure or property may be contained in at least one implementation of the present invention. “In one embodiment” appearing in different places in this specification does not mean the same embodiment, nor an embodiment that is exclusive or selective from other embodiments.

### EXAMPLE 1

Coconut oil and long-chain fatty acids (oleic acid: linoleic acid=2:1) were added to the reactor as a molar ratio of 1:3, adding 8wt % immobilized lipase NS 40086, and the temperature was kept at 60° Cstirring at 300 r/min for five hours. After the reaction, the oil was transferred to the centrifugal tube and centrifugated at 2000 r/min for 2min. Lipase and oil were separated, the alkali neutralization process removed free fatty acids in the oil, and fat composition 1 was obtained.

### EXAMPLE 2

The palm kernel oil and basa fish oil (5:5wt %) were mixed, melted, and added to the reactor. Adding 8% Lipase NS 40086, the temperature was kept at 60° C. for eight hours, stirring at of 300 r/min. After the reaction, the oil was transferred to the centrifugal tube and centrifuged at 2000 r/min for 2min. Lipase and oil were separated to obtain the fat composition 2.

### EXAMPLE 3

The palm kernel oil and extracted lard (3.5:6.5wt %) were mixed, melted, and added to the reactor. 10% Lipase NS 40086 was added; the temperature was kept at 60° C. for four hours under stirring at 400 r/min. After the reaction, the oil was transferred to the centrifugal tube and centrifuged at 2000 r/min for 2min. Lipase and oil were separated to obtain the fat composition 3.

TABLE 1
The Lipid composition of the three fats (%)

| Glycerides | | Fat 1 | Fat 2 | Fat 3 |
|---|---|---|---|---|
| Triacylglycerol | | 93.2 | 95.6 | 94.8 |
| Others | Diacy-lizlycerol | 4.1 | 2.3 | 3.6 |
| | Monoacylglycerol | 2.3 | 0 | 0 |
| | Free fatty acid | 0.4 | 2.1 | 1.6 |

### REFERENCE EXAMPLE

2.75 g of medium-chain triacylglycerol and 52.25 g of long-chain triacylglycerol (rapeseed oil) were mixed and added to the reactor, and the water in the raw oil was removed by vacuum drying at 95° C. for 30 min. Then, the temperature was kept at 75° C., 0.0825 g of sodium methoxide was added, and the reaction was terminated by adding citric acid solution after 35 min. The molar ratio of citric acid to sodium methoxide was 3:1, and the amount of water was 30% of the mass of the substrate. The crude MLCT fat was prepared by complete oscillation, centrifugation, washing, and vacuum drying. Then the fat composition 4 was prepared by refining.

The fat 1, 2, 3 were purified and then to determine the composition of fatty acid and triacylglycerol. The composition of fatty acids was determined by GC-FID (gas chromatography with flame ionization detector). Triacylglycerol composition was determined using ACQUITY UPC-MS (ultra-performance liquid chromatography with quadrupole time of flight tandem mass spectrometer).

The fatty acid composition of the four fats is shown in Table 2. The triacylglycerol composition of the four fats is shown in Table 3.

TABLE 2
Fatty acids composition of the fats (%)

| Fatty | (% of total fatty acid) | | | | (% of sn-2 fatty acid) | | | |
|---|---|---|---|---|---|---|---|---|
| acids | Fat 1 | Fat 2 | Fat 3 | Fat 4 | Fat 1 | Fat 2 | Fat 3 | Fat 4 |
| 8:0 | 2.52 | 2.57 | 2.20 | 6.32 | 3.08 | 1.12 | 1.08 | 5.21 |
| 10:0 | 2.79 | 2.52 | 2.79 | 4.54 | 6.01 | 2.85 | 3.01 | 3.74 |
| 12:0 | 23.32 | 21.47 | 22.32 | nd | 30.56 | 20.45 | 25.56 | nd |
| 14:0 | 11.24 | 10.39 | 11.08 | 0.20 | 10.85 | 14.34 | 16.85 | 2.66 |
| 16:0 | 16.92 | 18.45 | 17.92 | 4.08 | 34.42 | 48.36 | 38.42 | 20.06 |
| 18:0 | 3.53 | 3.89 | 3.82 | 2.84 | 1.89 | 2.00 | 3.89 | nd |
| ZSFA | 60.32 | 59.29 | 60.13 | 17.98 | 86.81 | 89.12 | 88.81 | 31.67 |
| 16:1 | 0.08 | 0.71 | 0.08 | nd | nd | nd | nd | nd |
| 18:1 | 31.06 | 28.61 | 30.66 | 69.72 | 7.16 | 8.03 | 6.16 | 53.10 |
| 18:2 | 8.39 | 10.96 | 8.98 | 10.82 | 6.03 | 2.85 | 5.03 | 14.21 |
| 18:3 | 0.15 | 0.20 | 0.15 | 0.43 | nd | nd | nd | 1.02 |
| 20:4 | nd | nd | nd | 0.78 | nd | nd | nd | nd |
| 20:5 | nd | 0.09 | nd | 0.27 | nd | nd | nd | nd |
| 22:6 | nd | 0.14 | nd | nd | nd | nd | nd | nd |
| SUFA | 3908 | 40.61 | 39.87 | 82.02 | 13.19 | 10.88 | 11.19 | 68.33 |

SFA, saturated fatty acid;
UFA, unsaturated fatty acid;
nd, not detected.

According to the table above, the mass ratios of long-chain fatty acids to medium-chain fatty acids of the fat 1, 2, 3, and 4 are 2.5:1, 2.8:1, 2.7:1, 8.2:1, respectively.

TABLE 3
Triacylglycerol composition of the fats (%)

| TAG | Fat 1 | Fat 2 | Fat 3 | Fat 4 |
|---|---|---|---|---|
| TAG containing three medium- | 5 | 9 | 2.9 | 2.93 |
| chain fatty acids | | | | |
| 8:0-8:0-10:0 | nd | nd | nd | 2.93 |
| 10:0-10:0-12:0 | 0.92 | 1.85 | 0.88 | nd |
| 10:0-12:0-12:0 | 1.77 | 2.33 | 0.89 | nd |
| 12:0-12:0-12:0 | 2.31 | 4.82 | 1.22 | nd |
| TAG containing two medium- | 10 | 20 | 5.1 | 0.96 |
| chain fatty acids | | | | |
| 8:0-8:0-18:2 | nd | nd | nd | 0.35 |
| 8:0-8:0-18:1 | nd | nd | nd | 0.19 |
| 10:0-10:0-14:0 | 0.62 | 2.22 | 1.1 | 0.42 |
| 10:0-12:0-18:1 | 2.61 | 5.12 | 1.12 | nd |
| 10:0-12:0-18:2 | 1.93 | 3 | 0.3 | nd |
| 12:0-12:0-18:1 | 3.08 | 5.89 | 1.56 | nd |
| 12:0-12:0-14:0 | 1.76 | 3.77 | 1.02 | nd |
| TAG containing one medium- | 75 | 60 | 90 | 50.96 |
| chain fatty acid | | | | |
| 8:0-18:1-18:1 | nd | nd | nd | 11.48 |
| 8:0-14:0-18:2 | nd | nd | nd | 4.43 |
| 8:0-14:0-18:1 | nd | nd | nd | 8.31 |
| 10:0-18:1-18:1 | nd | 0.30 | 0.41 | 11.16 |
| 10:0-18:1-18:2 | nd | 0.21 | 0.10 | 6.29 |
| 10:0-18:1-18:0 | nd | 1.88 | nd | 5.02 |
| 10:0-16:0-18:2 | 5.57 | 3 | 6.11 | 1.02 |
| 10:0-14:0-18:2 | 2.76 | 2.22 | 3 | 3.25 |
| 10:0-16:0-18:1 | 6.39 | 4.41 | 7.73 | nd |
| 12:0-14:0-14:0 | 1.42 | 0.67 | 2.88 | nd |
| 12:0-16:0-18:1 | 16.12 | 13.86 | 19 | nd |
| 12:0-16:0-14:0 | 4.11 | 2 | 5.65 | nd |
| 12:0-18:1-18:1 | 12.29 | 12 | 15.88 | nd |
| 12:0-18:1-18:2 | 11.42 | 9.32 | 14.2 | nd |
| 12:0-18:2-18:2 | 5.49 | 3.13 | 6 | nd |
| 12:0-16:0-18:2 | 9.43 | 7 | 9.04 | nd |
| TAG containing none of medium- | 10 | 11 | 2 | 45.15 |
| chain fatty acids | | | | |
| 14:0-18:1-18:2 | 3 | 2.27 | nd | nd |
| 14:0-16:0-18:1 | 2.17 | 3.5 | 0.09 | nd |
| 16:0-16:0-18:0 | 0.10 | nd | nd | 2.17 |
| 16:0-16:0-18:1 | 0.97 | 0.88 | nd | 3.37 |
| 16:0-16:0-18:2 | 1.03 | 1 | nd | nd |
| 16:0-18:0-18:1 | 0.82 | 0.59 | nd | 4.60 |
| 16:0-18:1-18:1 | 0.9 | nd | 1.2 | 8.92 |
| 16:0-18:1-18:2 | 0.3 | 0.6 | 0.71 | 3.42 |
| 16:0-18:2-18:2 | nd | 1.05 | nd | nd |
| 18:0-18:1-18:1 | nd | 0.11 | nd | 9.80 |
| 18:1-18:1-18:2 | 0.50 | 1 | nd | 12.87 |
| 18:1-18:1-18:1 | 0.21 | nd | nd | nd |

TAG, triacylglycerol;
nd, not detected.

### EXAMPLE 4

### Determination of Oxidation Stability

The oxidation stability of oil and lipid is related to the product's processability, storage, and shelf life. The oxidation induction time of the fat composition 1, 2, 3, and 4 was measured using a Rancimat instrument (Omnionion OSI, American) according to the national standard GB/T21121. 3 g sample was weighed and placed in a unique test tube. 0.038 MPa dry air was introduced into the test tube at a flow rate of 10 L/h at 110° C.±0.1° C. Then the induction time of oxidation of the fat products was determined.

TABLE 4
Comparison of oxidation induction time of the fats

| | | | | Reference |
|---|---|---|---|---|
| | Embodiment examples | | | example |
| | Fat 1 | Fat 2 | Fat 3 | Fat 4 |
| Induction time/h | 6.9 + 0.1* | 7.1 + 0.2* | 5.5 + 0.2 | 4.0 + 0.3 |

*represents significant difference at the level of 0.05.

According to the table above, the oxidation induction time of the fat composition 4 is significantly lower than that of the fat composition 1, 2, 3. It indicates the fat composition 1, 2, and 3 have better oxidation stability.

### EXAMPLE 5

### Determination of Lipid Metabolism

Specified pathogen-free grade Wistar rats (1-week-old, male) were kept in the clean experimental animal centre at 23±2° C., the humidity of 60%, and drinking water and food are available. The care and pretreatment of experimental animals are carried in accordance with the relevant provisions of the Regulations on the Management of Experimental Animals. Twenty-eight male Wistar rats were randomly divided into four groups, including a control group, experimental group 1, experimental group 2 and experimental group 3 after a week of adjustable feeding a basal diet. The diet of each group was formulated according to the formula of experimental animal feed recommended by the American Society for Nutrition. The dietary fat in the basal control group was the fat composition 4 replacing the fat of basal diet, and the fat in the three experimental groups was the fat composition 1, 2, and 3 respectively, replacing the fat of basal diet. The packing and drinking water were changed every day. After 1 week, the rats were fed separately in the metabolic cage for another 2 weeks. The food intake and feces of the rats in each group were recorded in the last 5 days.

TABLE 5
Basic dietary formula for rats

| | Experi- | Experi- | Experi- | |
|---|---|---|---|---|
| | mental | mental | mental | Control |
| Composition | group 1 | group 2 | group 3 | group |
| Fat | 20 g | 20 g | 20 g | 20 g |

| | | |
|---|---|---|
| Others | Sucrose | 10 g |
| | Casein | 25 g |
| | Corn starch | 20 g |
| | Maltodextrin | 15 g |
| | Cysteine | 0.25 g |
| | Cellulose | 5 g |
| | Choline | 0.25 g |
| | bitartrate | |
| | Vitamins | L g |
| | Minerals | 3.5 g |

The total lipid in the faeces was extracted after being dried and weighed. The fat absorption rate (%)=(fat intake—fat in feces)/fat intake×100.

Blood samples were collected after 4 weeks of feeding. After being fasted for 12 h, the rats were weighed and killed by heart puncture. The plasma triacylglycerol (TG), cholesterol (TC), high-density lipoprotein cholesterol (HDL-C), and low-density lipoprotein cholesterol (LDL-C) were measured.

TABLE 6
Weight Gain, fat excretion and absorption of the rats

| | Experimental | | | Control |
|---|---|---|---|---|
| | group 1 | Experimental | Experimental | group |
| weight | 15.54 ± 0.64 | 16.18 ± 0.32 | 15.38 ± 0.71 | 17.38 ± 0.27 |
| Gain (g) | | | | |
| faecal | 230 ± 12 | 238 ± 3.3 | 226 ± 5.83 | 246 ± 10 |
| fat (mg) | | | | |
| Fat | 90.32 ± 1.88 | 89.80 ± 0.97 | 90.12 ± 1.25 | 80.12 ± 1.02 |
| absorption | | | | |
| (%) | | | | |

TABLE 7
Plasma TG, TC, HDL-C and LDL-C levels of the rats

| | | Experimental | Experimental | Control |
|---|---|---|---|---|
| | Experimental | group 2 | group 3 | group |
| TG (mmol/L) | 0.42 ± 0.15 | 0.5H0.04 | 0.58 ± 0.20 | 0.78 ± 0.22 |
| TC (mmol/L) | 3.3 ± 0.80 | 4.70 ± 0.50 | 4.04 ± 0.08 | 6.04 ± 0.28 |
| HDL-C (mmol/L) | 2.16 ± 0.20 | 1.89 ± 0.30 | 2.02 ± 0.23 | 3.02 ± 0.11 |
| LDL-C (mmol/L) | 0.14 ± 0.05 | 0.25 ± 0.11 | 0.28 ± 0.10 | 0.78 ± 0.07 |

TG, triacylglycerol;
TC, cholesterol;
HDL-C, high-density lipoprotein cholesterol;
LDL-C, low-density lipoprotein cholesterol.

The results show that the rats are expected to have an excellent mental state, smooth fur, and normal defecation during the feeding process. Table 6 shows that the fat absorption rates of experimental groups 1, 2, and 3 are higher than those of the control group. Table 7 shows that plasma TG and TC contents in the control group were higher than in experimental groups 1, 2, and 3. All the results indicate that the fat compositions provided by the invention can effectively improve the absorption rate and utilization of fat without increasing the levels of blood TC, TG, and LDL-C.

### EXAMPLE 6

### Determination of the in Vitro Digestibility

Preparation of emulsions with different fats: an amount of emulsifier whey protein was dissolved in ultrapure water and completely dissolved and hydrated by stirring in a constant temperature water bath for more than four hours. 97% of whey protein was mixed with 3% the fat composition (fat 1-4) for 5 min, then emulsified for 3 min by high-speed shear dispersing, and homogenized under 20 MPa to prepare emulsion 1-4.

The in vitro digestion of infants:

Preparation stage: the digestion temperature was set as 37° Cr; the initial pH of the stomach was adjusted to 2.7, and 2 mL of simulated gastric juice and 9.1 mg of rabbit gastric lipase were added into the gastric chamber; the initial pH of the small intestine was adjusted to 6.2, and 5 mL of 1.6 mmol/L bile and 5 mL of 10% pancreatic enzyme were added to the intestinal digestive chamber.

Digestion stage: 35 mL emulsion was added into the gastric digestion chamber and entered the retention stage for 10 min. At the same time, opened the magnetic stirrer (intermittent mode, 100 rpm), acid pump and alkali pump (control the real-time change of pH in gastric digestive chamber and maintain the pH in intestinal digestive chamber at 6.2), and gastric juice pump (adding gastric juice, 1 mL/min).

Emptying stage: the gastric juice pump speed was adjusted to 0.5 mL/min, then the pancreatic fluid pump and bile pump was opened, and the infusion rate was kept as 0.25 mL/min and 0.5 mL/min, respectively. Next, the gastric emptying pump and intestinal transport pump were opened to stimulate gastric emptying and intestinal transport.

The digestion lasted 160 min and was carried out in triplicate. The digested samples were collected from the digestive chamber every 20 minutes before and after digestion, and hydrochloric acid (6 mol/L) was added immediately to inactivate the enzyme after the collection. The lipid was extracted immediately.

Lipid extraction: 1 mL ammonia water was added into samples and then mixed in a water shaking bath at 65±5° C. Then, 5 mL absolute ethanol, 12.5 mL absolute ether, and 12.5 mL petroleum ether were added to extract the lipids. The samples were mixed thoroughly and stood, and then the supernatants were collected. The lipids in the lower phase were extracted using half of the solvents as above. The solvents were removed by nitrogen, and the lipids were stored in a −20° C. freezer until analysis.

Determination of lipid composition: the lipid simple (10 mg) was prepared at a concentration of 20 mg/mL dissolved in n-hexane, and the supernatant was filtered and analyzed by liquid chromatography.

The samples were analyzed by liquid chromatograph equipped with a refractive index detector and a Sepax HP-Silica column (4.6 mm×250 mm×5 μm). The column temperature was 35° C. The mobile phase was n-hexane, isopropanol, and formic acid (15:1:0.3, v/v/v) with a flow rate of 1 mL/min. Moreover, the injection volume of each sample was 20 μL.

The lipid of the digested sample included residual TAG, DAG, MAG, and FFA. The mass data were converted to molar data employing average molar mass (g/mol) calculated from the fatty acid composition of the fat composition or human milk. The degree of lipolysis at a certain point in the digestion process was expressed as the molar percentage of FFA to the sum of FFA and residual triacylglycerol acyl chain.

$$Digestibility = \frac{100 * [ FFA ]}{( 3 * [ TAG ] + 2 * [ DAG ] + [ MAG ] + [ FFA ]}$$

([FFA], [TAG], [DAG], and [MAG] is the average molar mass of free fatty acids, triacylglycerol, diacylglycerol, and monoacylglycerol, g/mol)

### RESULTS

According to the digestibility change diagram of the fat composition 1, 2, 3, 4, and human milk during in vitro simulated digestion, it can be seen that the digestibilities of the fat compositions 1 to 4 are similar during gastric digestion, and they are slightly lower than that of human milk after 120 min digestion in the stomach. After intestinal digestion, the digestibilities of the fat composition 1, 2, and 3 are significantly higher than that of the fat composition 4. Compared with fat composition 4, the digestibilities of the fat compositions 1-3 are closer to that of human milk. The digestibility of the fat composition 4 is high only in the stomach but low in the small intestine, and its slow energy supply may affect the development of the body.

Infants are at a unique growth stage with an immature digestive and absorption system, which is different from that of adults. The pH (-5) of gastric is high, but the digestive enzyme activity and bile salt content are low, and the absorption rate of lipid is lower than that of adults. Triacylglycerols are hydrolyzed preliminary in the stomach by means of gastric lipase. Gastric lipase, an sn-1, 3-position specific enzyme, whose optimal pH is 3-5, hydrolyzes sn-3 fatty acids preferentially and hydrolyzes triacylglycerol into sn-1, 2- diacylglycerol, and free fatty acids. The fat and its hydrolysates are drained into the duodenum after gastric lipase initial digestion and then hydrolyzed by pancreatic lipase. Pancreatic lipase is an sn-1, 3-position specific enzyme that hydrolyzes undigested fats into sn-2 monoacylglycerol and free fatty acids, which are then transported to the brush margin of the intestinal mucosa and absorbed by the intestinal epithelial cells. Compared with the long-chain saturated fatty acids, short- and medium-chain fatty acids and unsaturated fatty acids are more likely to be absorbed by small intestine epithelial cells. Because the melting point of free long-chain saturated fatty acids is high, they are easy to form insoluble calcium soaps with calcium and other metal ions in the small intestine, which are excreted in the stool, leading to the loss of fat and calcium and causing the stool of infants to harden or constipation. Long-chain saturated fatty acids can be directly absorbed by the small intestine in the form of sn-2 monoacylglycerol, thus improving the absorption rate of fat and reducing the loss of calcium. Therefore, the structure of triacylglycerols plays a crucial role in the growth and development of infants.

The differences in digestibility of the four fat compositions are mainly related to the composition of fatty acids and the structure of triacylglycerols. Medium-chain triacylglycerols can be hydrolyzed by lipase quickly, and the released medium-chain fatty acids are absorbed quickly in the body. In contrast, long-chain triacylglycerols were hydrolyzed and released slowly, which would increase the gastrointestinal burden. The MLCT based fats (1, 2, 3) of the invention include medium-chain fatty acids And the saturated fatty acids are mainly distributed in the sn-2 position, which are easy to be absorbed. Although fat composition 4 also contains medium-chain fatty acids, it is mainly 8:0 and 10:0 and is quickly hydrolyzed in the stomach. However, its saturated fatty acids are mainly distributed in the sn-1,3 position. Under the alkaline environment of the small intestine, it is easy to form insoluble calcium soap with calcium and other metal ions, which affects the hydrolysis of triacylglycerols by lipase and leads to the decrease of fat digestibility.

The fat composition enriched with medium-and long-chain triacylglycerol of the invention contains both medium-chain fatty acids and long-chain fatty acids. Furthermore, sn-2 saturated fatty acids reach more than 60%. It provides medium-chain fatty acids and ensures the requirements of high saturated fatty acids at the sn-2 position. It has the advantages of good oxidation stability, improving the fat absorption rate of rats and in vitro digestibility, and ensuring energy supply, showing a favorable effect on the growth and development of infants.

It should be stated that the above examples are used only to illustrate the procedures of this invention, but not the limitation to them. Those skilled in this art also will appreciate that while the above examples illustrate the application of this invention, the process is applicable to essentially all substances containing lipids. Slight variations in procedures may well present themselves to those skilled in this art without detracting from the scope and spirit of the invention described herein.

1. A fat composition enriched with medium-and long-chain triacylglycerols, said composition comprising:
in terms of fatty acid composition of the fat composition, the content of medium-chain fatty acids and long-chain fatty acids reach above 99%, the content of short-chain fatty acids is less than 1%, and the mass ratio of long-chain fatty acids to medium-chain fatty acids is from 1.2:1 to 3:1; there are not only medium-chain fatty acids but also long-chain fatty acids at the sn-2 position, where the content of saturated fatty acids is more than 60%;
in terms of triacylglycerol composition of the fat composition, a triacylglycerol containing one medium-chain fatty acid and two long-chain fatty acids makes up 50-90% of the total triacylglycerol, a triacylglycerol containing two medium-chain fatty acid and one long-chain fatty acid makes up 3-35% of the total triacylglycerol, a triacylglycerol containing three medium-chain fatty acids makes up 0-10% of the total triacylglycerol
the short-chain fatty acids comprise at most 6 carbon atoms; the medium-chain fatty acids comprise 6-12 carbon atoms; the long-chain fatty acids comprise 14-24 carbon atoms.

2. The fat composition of claim 1 wherein the medium-chain fatty acids comprise 10-12 carbon atoms; the long-chain fatty acids comprise 16-18 carbon atoms.

3. The fat composition of claim 1 wherein the medium-chain fatty acids comprise lauric acid (La).

4. The fat composition of claim 1 wherein the long-chain fatty acids comprise palmitic acid (P), oleic acid (O), linoleic acid (L).

5. The fat composition of claim 1 wherein the medium-and long-chain triacylglycerols comprise LaPO, LaOO, LaOL, LaLL, and LaPL.

6. The fat composition of claim 1, wherein LaPO makes up 10-60% of the fat composition, LaOO makes up 5-40% of the fat composition, LaOL makes up 5-30% of the fat composition, LaLL makes up 0-10% of the fat composition, LaPL makes up 0-10% of the fat composition.

7. The fat composition of claim 1 wherein medium-chain fatty acids and long-chain fatty acids are derived from one or more fats of vegetable oil or animal oil or its extracts or milk fat.

8. The fat composition of claim 1 wherein the fat composition can be used as an additive in food.

9. The fat composition of claim 8 wherein the fat composition can be added with a concentration of 15-50% to infant formula, follow-on formula, or infant food.

10. A process of making a fat composition of claim 1 comprising mixing the medium-chain triacylglycerol and the long-chain fatty acids with the ratio of 1:2-1:5 (mol/mol), or the long-chain triacylglycerol and the medium-chain triacylglycerol with the ratio of 1:0.5-1:2 (mol/mol), adding lipase with a mass ratio of 4˜14% to the substrate at 40˜65 ° C., and reacting for 0.5˜10 hours under solvent-free conditions.

11. A process of making a fat composition of claim 1 comprising mixing the medium-chain triacylglycerol and the long-chain triacylglycerol with the ratio of 1:0.5 to 1:3, adding lipases with a mass ratio of 4˜14% to the substrate at 40˜65° C., and reacting for 0.5˜12 hours under solvent-free conditions.

12. The process of claim 10 wherein the medium-chain triacylglycerols comprise one or more kinds of fats such as coconut oil, coconut oil extract, palm kernel oil, palm kernel oil extract, laurin, tricaprylin, or tricaprin.

13. The process claim 10 wherein the medium-chain triacylglycerols comprise glycerides containing caprylic acid, or capric acid, or lauric acid.

14. The process of claim 10 wherein the medium-chain fatty acids comprise one or more fatty acids such as caprylic acid, capric acid, lauric acid.

15. The process of claim 10 wherein the long-chain fatty acids comprise one or more fatty acids such as myristic acid, palmitic acid, oleic acid, linoleic acid, stearic acid, linolenic acid, arachidonic acid, eicosapentaenoic acid, docosapentaenoic acid, docosahexaenoic acid.

16. The process of claim 10 wherein the long-chain triacylglycerols comprise fish oil, lard oil, or extracts.

17. The process of claim 10 or 11 wherein the lipases comprise one or more of immobilized enzyme NS 40086, Lipozyme TL IM, Lipozyme RM IM, Lipozyme 435, Novozym 435, Lipase PS, Lipase AK, Lipase AH, and Lipase AYS.

18. The fat composition of claim 1 wherein LaPO makes up 10-45% of the fat composition, LaOO makes up 5-25% of the fat composition, LaOL makes up 5-20% of the fat composition, LaLL makes up 0-5% of the fat composition, LaPL makes up 0-5% of the fat composition.

19. The use of fat composition of claim 8 wherein the fat composition can be used as the additive in food for infants and children.
